(19) United States
(12) Patent Application Publication
(10) Pub. No.: US 2022/0022768 A1
(43) Pub. Date: Jan. 27, 2022

(54) FLUID MONITORING DEVICE INCLUDING IMPEDANCE SENSING ELEMENT

(71) Applicant: 3M INNOVATIVE PROPERTIES COMPANY, St. Paul, MN (US)

(72) Inventors: Myungchan Kang, Woodbury, MN (US); Jaewon Kim, Woodbury, MN (US); Jung-Ju Suh, Seoul (KR); Chekhua Chua, Shanghai (CN)

(21) Appl. No.: 17/309,591

(22) PCT Filed: Dec. 10, 2018
(86) PCT No.: PCT/CN2018/120064

§ 371 Date: Jun. 8, 2021

(51) Int. Cl. A61B 5/053; A61B 5/00; A61M 5/00; A61M 5/14

(52) U.S. Cl. CPC A61B 5/053; A61B 5/6833; A61M 5/002; A61B 2562/227; A61M 2205/3389; A61B 2562/18; A61B 2562/164; A61M 5/14

(57) ABSTRACT

Fluid monitoring devices (100,200,700) including an impedance sensing element (110,210,410,510,610,710,61,62,63) are provided. The impedance sensing element (110,210,410,510,610,710,61,62,63) includes a calibration portion (212, 412, 512, 612, 712) and a measurement portion (214,414,514,614,714), and the fluid monitoring devices (100,200,700) can be self-calibrated in real time based on calibration data from the calibration portion (212,412,512,612,712).

### TECHNICAL FIELD

The present disclosure relates to fluid monitoring devices including impedance sensing elements, and methods of making and using the devices.

### BACKGROUND

Acoustic resonance sensors and optical sensors are widely used for monitoring liquid level in an infusion line of an intravenous (IV) therapy. Such commonly used sensors are expensive and complex.

### SUMMARY

The present disclosure describes fluid monitoring devices including impedance sensing elements, and methods of making and using the sensing devices.

In one aspect, the present disclosure describes a flexible sensor for fluid monitoring. The sensor includes a flexible substrate having a first side and a second side opposite the first side; an impedance sensing element disposed on the first side of the flexible substrate; and a circuit unit functionally connected to the sensing element to receive data related to an impedance of the impedance sensing element from the impedance sensing element and process the data. The impedance sensing element includes a calibration portion and a measurement portion electrically connected to the calibration portion, the calibration portion configured to generate calibration data, and the measurement portion configured to generate measurement data. The circuit unit is configured to calibrate the measurement data based on the calibration data.

In another aspect, the present disclosure describes a method of monitoring fluid. The method includes providing an impedance sensing element including a calibration portion and a measurement portion electrically connected to the calibration portion; disposing the impedance sensing element adjacent to a volume of fluid to be monitored; varying the fluid volume such that a fluid level thereof continuously runs across the calibration portion and the measurement portion of the sensor in sequence; and measuring an impedance-related property of the impedance sensing element when varying the fluid volume to obtain a plot of impedance-related property versus fluid level. The plot has a calibration segment corresponding to the calibration portion of the sensor and a measurement segment corresponding to the measurement portion of the impedance sensing element. The calibration segment and the measurement segment are connected at a transitional point. In some embodiments, the method further includes calibrating, via a circuit unit, the impedance sensing element based on the calibration segment of the plot.

Various unexpected results and advantages are obtained in exemplary embodiments of the disclosure. One such advantage of exemplary embodiments of the present disclosure is that the flexible sensor described herein can be self-calibrated upon measuring different fluids having different dielectric properties. Also, the flexible sensors use impedance sensing elements which are relatively low-cost and simpler as compared to typical acoustic resonance sensors and optical sensors. Some flexible sensors may have a symmetric configuration to exhibit an orientation-independent performance.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1A illustrates a schematic side view of a fluid monitoring device including impedance sensing element attached to an intravenous (IV) bag, according to one embodiment.

FIG. 1B illustrates a cross-sectional view of the fluid monitoring device of FIG. 1A, according to one embodiment.

FIG. 1C illustrates a plot of impedance versus fluid level for the fluid monitoring device of FIG. 1A.

FIG. 1D illustrates a plot of admittance, capacitance, or conductance versus fluid level for the fluid monitoring device of FIG. 1A for monitoring different fluids.

FIG. 2A illustrates a schematic side view of a fluid monitoring device including impedance sensing elements attached to an intravenous (IV) bag, according to one embodiment.

FIG. 2B illustrates a plot of admittance, capacitance, or conductance versus fluid level for the fluid monitoring device of FIG. 2A.

FIG. 2C illustrates a plot of admittance, capacitance, or conductance versus fluid level for the fluid monitoring device of FIG. 2A for monitoring different fluids.

FIG. 3 illustrates a flow diagram of a method to monitoring a fluid level, according to one embodiment.

FIG. 4A illustrates a schematic side view of a fluid monitoring device, according to one embodiment.

FIG. 4B illustrates a schematic side view of a fluid monitoring device, according to another embodiment.

FIG. 4C illustrates a schematic side view of a fluid monitoring device, according to another embodiment.

FIG. 5A illustrates a schematic side view of a fluid monitoring device including a rectangular-shaped impedance sensing element, according to one embodiment.

FIG. 5B illustrates a plot of admittance, capacitance, or conductance versus fluid level for the fluid monitoring device of FIG. 5A.

FIG. 6A illustrates a schematic side view of a fluid monitoring device including a symmetric-shaped impedance sensing element, according to one embodiment.

FIG. 6B illustrates a schematic side view of a fluid monitoring device including a symmetric-shaped impedance sensing element, according to another embodiment.

FIG. 6C illustrates a schematic side view of a fluid monitoring device including a symmetric-shaped impedance sensing element, according to another embodiment.

FIG. 6D illustrates a plot of admittance, capacitance, or conductance versus fluid level for the fluid monitoring device of FIG. 6A, 6B or 6C.

FIG. 7A illustrates a schematic side view of a fluid monitoring device including a round-shaped impedance sensing element.

FIG. 7B illustrates a plot of admittance, capacitance, or conductance versus fluid level for the fluid monitoring device of FIG. 7B.

FIG. 8 illustrates a schematic diagram of a fluid monitoring device wirelessly connected to a mobile device, according to one embodiment.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure provides fluid monitoring devices including impedance sensing elements, and methods of making and using the devices.

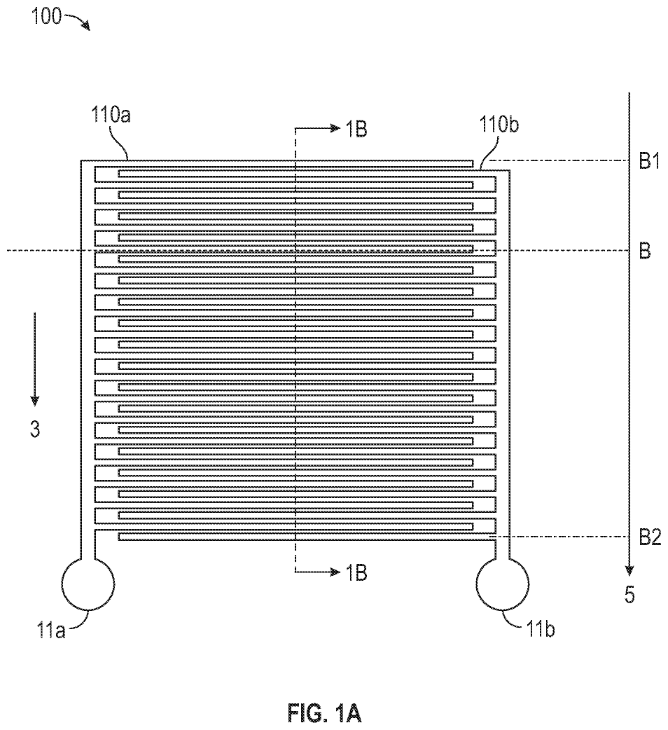

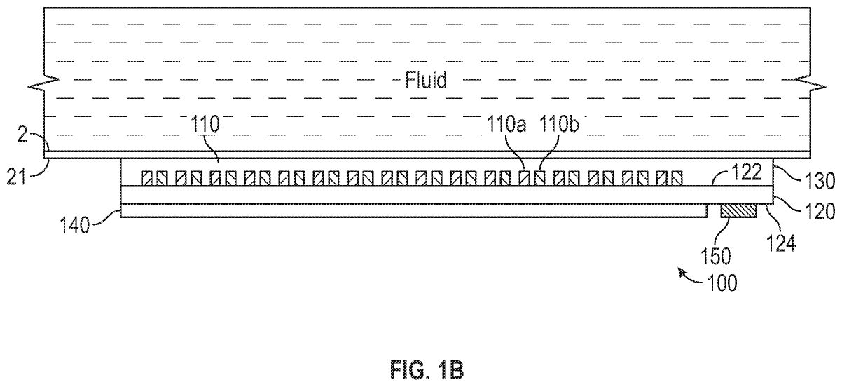

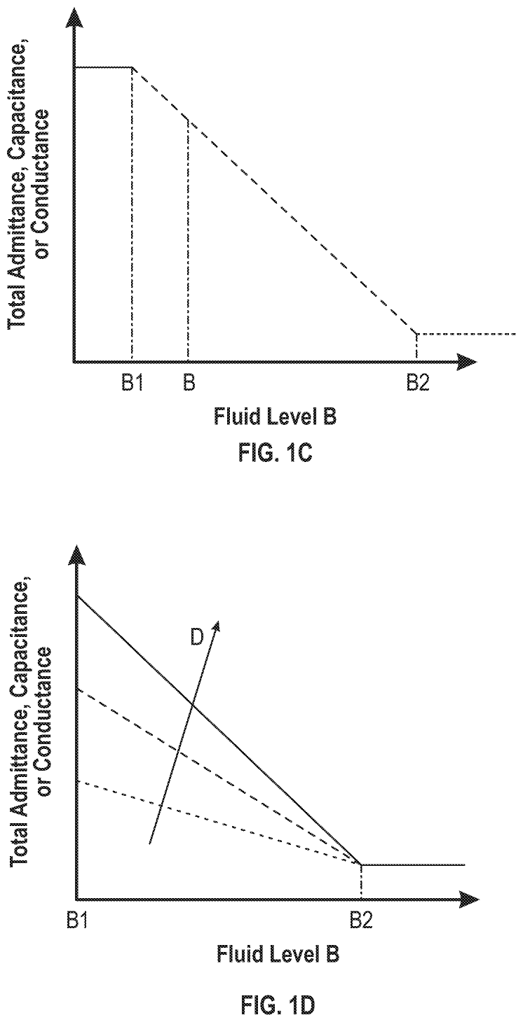

FIGS. 1A-B illustrate a fluid monitoring device 100 including impedance sensing element 110 attached to an outer side 21 of a fluid container 2, according to one embodiment. The fluid monitoring device 100 includes an impedance sensing element 110 disposed on a first side 122 of a flexible substrate 120. The flexible substrate 120 can be made of any suitable insulative material such as, for example, a polymeric material. In some embodiments, the substrate 120 can be stretchable and bendable.

The fluid monitoring device 100 further includes an adhesive layer 130 on the first side 122 of the flexible substrate 120, configured to attach the device 100 to a fluid container 2 such as, for example, an intravenous (IV) bag. In some embodiments, an optional encapsulating layer can be provided between the adhesive layer 130 and the flexible substrate 120 to protect the impedance sensing element 110 and/or other circuitries on the flexible substrate 120. The optional encapsulating layer can be, for example, a polymeric layer or other suitable coating layers to prevent direct moisture contact to the impedance sensing element 110. A releasable liner can be used to protect the adhesive surface of the adhesive layer 130 before use. In some embodiments, the fluid monitoring device 100 includes an optional shielding layer 140 on the second side 124 of the flexible substrate 120, configured to shield electromagnetic interference (EMI) from the impedance sensing element 110. The shielding layer 140 may be made of any electrically conductive materials such as, for example, copper, transparent conductors, etc.

In the depicted embodiment of FIGS. 1A-B, the impedance sensing element 110 includes a pairing of interdigitated electrode or finger arrays 110a and 110b connected to connection pads 11a and 11b, respectively. The finger arrays 110a and 110b are arranged interdigitated and parallel with respect to each other to produce a capacitor-like, high pass filter characteristic. An impedance-related property of the impedance sensing element 110 can be determined by various factors such as, for example, a configuration of the finger arrays 110a and 110b, a dielectric property of a fluid contained in the fluid container 2, etc. An impedance-related property may include, for example, impedance, admittance, conductance, capacitance, dissipation factor, phase angle, etc. The configurations of the finger arrays 110a and 110b may include, for example, a finger length, a distance between adjacent fingers, etc. The impedance sensing element 110 extends along an elongation direction 3 between positions B1 and B2.

It is to be understood that an impedance sensing element described herein can be any suitable impedance sensing element other than an interdigitated capacitor as long as it can monitor the adjacent fluid by measuring its impedance-related property. For example, in some embodiments, the impedance sensing element may include one or more parallel-plates capacitors or other suitable types of capacitors.

The device 100 further includes a circuit unit 150 functionally connected to the sensing element 110 to receive data related to an impedance of the impedance sensing element 110 from the impedance sensing element 110 and process the data to obtain fluid-volume-related information. In some embodiments, the circuit unit 150 may include a microprocessor to process the data. In some embodiments, the circuit unit 150 may include a wireless component such as, for example, a Bluetooth Low Energy (BLE) component. It is to be understood that a fluid monitoring device described herein can integrate with any suitable functional circuitry to make use of an impedance sensing element thereof.

When the fluid monitoring device 100 is attached to the outside 21 of the fluid container 2, the impedance sensing element 110 is oriented with its elongation direction 3 substantially parallel to a vertical direction 5, substantially perpendicular to a fluid level B of the fluid inside the container 2, as shown in FIG. 1A. An impedance-related property (e.g., impedance, admittance, capacitance, conductance, etc.) of the sensing element 110 can be measured upon the variation of fluid level B along the vertical direction 5. One exemplary plot of admittance, capacitance, or conductance versus fluid level for the fluid monitoring device 100 of FIG. 1A is shown in FIG. 1C. When the fluid volume inside the container 2 decreases and the fluid level B gradually changes from the position B1 to the position B2, the admittance, capacitance, or conductance of the sensing element 110 decreases accordingly. The fluid level, fluid volume, or fluid flow rate in the fluid container can be determined by measuring the impedance-related property of the impedance sensing element 110 via, for example, a plot of admittance, capacitance, or conductance versus fluid level.

The measured plots may vary, for example, depending on the dielectric property of the fluid contained in the fluid container. As shown in FIG. 1D, for a fluid having a higher dielectric constant, the admittance, capacitance, or conductance versus fluid level plot may have a greater slope (as indicated by the arrow D). In some embodiments, the fluid in the fluid container may be unknown. The plots in FIG. 1C may need to be calibrated first in order to determine the fluid volume or fluid level in the fluid container.

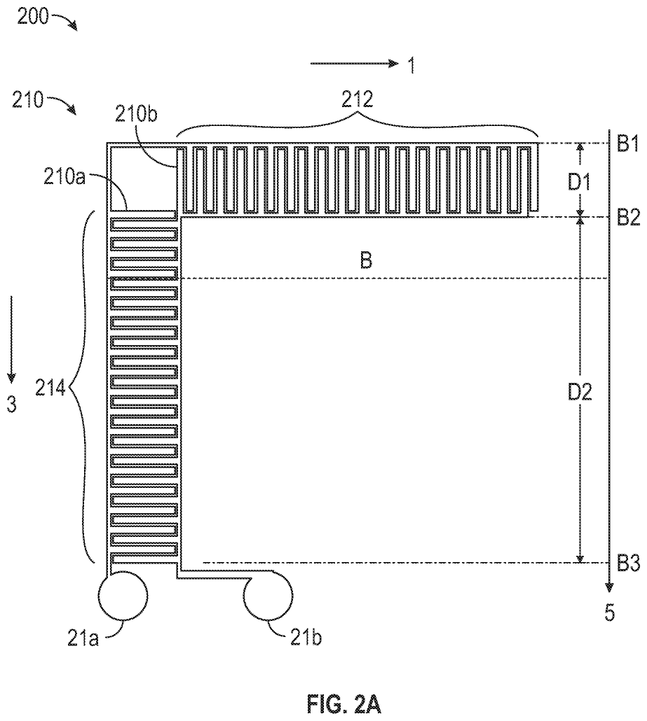

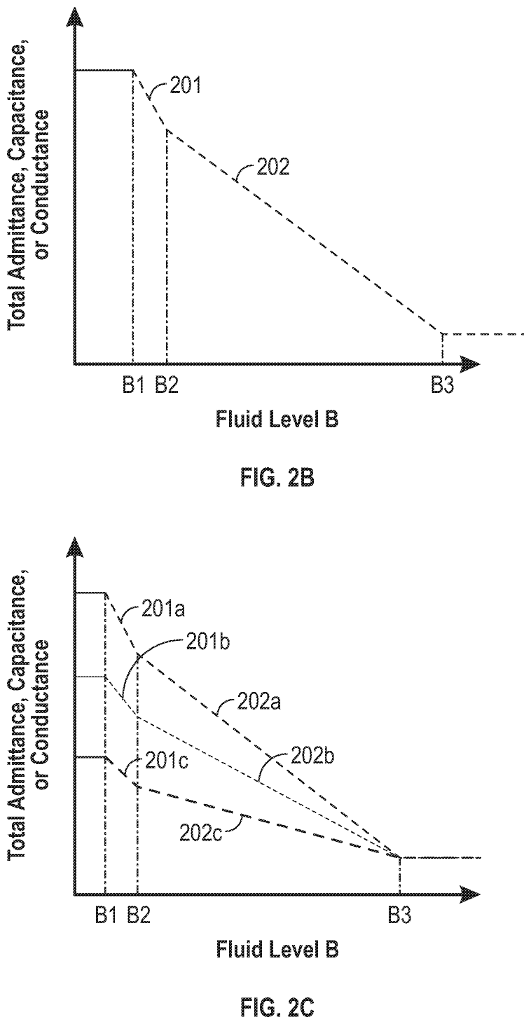

FIG. 2A illustrates a schematic side view of a fluid monitoring device 200 including an impedance sensing element 210 having a calibration portion 212 and a measurement portion 214 electrically connected to each other, according to one embodiment. The impedance sensing element 210 includes a pairing of interdigitated electrode or finger arrays 210a and 210b connected to connection pads 21a and 21b, respectively. The fingers 210a and 210b are arranged interdigitated and parallel with respect to each other to produce a capacitor-like, high pass filter characteristic. The calibration portion 212 of the sensing element 210 includes a first portion of the fingers 210a and 210b and extends along a lateral direction 1; the measurement portion 214 of the sensing element 210 includes a second portion of the fingers 210a and 210b and extends along the elongation direction 3. In the depicted embodiment of FIG. 2A, the lateral direction and the elongation direction are substantially orthogonal with respect to each other.

When the fluid monitoring device 200 is attached to an outside of a fluid container containing fluid, the impedance sensing element 210 is oriented such that the calibration portion 212 is substantially along a horizontal direction and the calibration portion 214 is substantially along a vertical direction. The calibration portion 212 and the measurement portion 214 form an up-side-down “L” shape. Along the vertical direction 5, the calibration portion 212 extends between positions B1 and B2 with a vertical length D1, and the measurement portion 214 extends between positions B2 and B3 with a vertical length D2. In some embodiments, the ratio of the vertical length D1 over the vertical length D2 may be in the range, for example, 0.01 to 1. A relatively short vertical length D1 can help to quickly calibrate the sensing element, while a relatively long vertical length D2 can provide an elongated window to quantitively monitor the fluid level.

An impedance-related property (e.g., impedance, admittance, capacitance, conductance, etc.) of the impedance sensing element 210 can be measured upon the variation of the fluid level B along the vertical direction 5. FIG. 2B illustrates an exemplary plot of admittance, capacitance, or conductance versus fluid level for the fluid monitoring device 200 of FIG. 2A that is attached to an outside of a fluid container. When the fluid level B runs across the calibration portion 212, i.e., changes from the position B1 to the position B2, the capacitance of the sensing element 110 decreases accordingly. The segment 201 of the plot between positions B1 and B2 corresponds to the calibration portion 212 of the impedance sensing element 210 and has a slope S1. When the fluid level B continues to run across the measurement portion 214, i.e., changes from the position B2 to the position B3, the capacitance of the sensing element 210 continues to decrease accordingly. The segment 202 of the plot between positions B2 and B3 corresponds to the measurement portion 214 of the impedance sensing element 210 and has a slope S2.

For a given fluid to be measured, the slopes S1 and S2 of the segments 201 and 202 can be determined by the configurations of the respective portions 212 and 214. In the depicted embodiment, the portions 212 and 214 have different orientations and produce segments having different slopes S1 and S2, where the position B2 is a transitional point connecting the calibration portion 212 and the measurement portion 214, and the slope changes from S1 to S2 across the transitional point. In some embodiments, S1 can be greater than S2, and the ratio of S1/S2 can be in the range of, for example, about 1 to about 10.

The fluid level or volume in the fluid container can be determined in real time based on the measured impedance-related property (e.g., impedance, admittance, capacitance, conductance, etc.) versus fluid level plot having a calibration segment and a measurement segment such as, for example, the plot of FIG. 2B. In some embodiments, the fluid monitoring device 200 can be calibrated by using the calibration segment 201 of the plot corresponding to the calibration portion 212 of the impedance sensing element 210. During the calibration, the dielectric property of the fluid inside the fluid container can be determined. With the calibration, the fluid level or volume in the fluid container can be determined in real time by using the measurement segment 202 of the plot when the fluid level B runs across the measurement portion 214.

The fluid monitoring device 200 can be used to determine a fluid level or volume of an unknown fluid in the fluid container. FIG. 2C illustrates plots of impedance-related property (e.g., impedance, admittance, capacitance, conductance, etc.) versus fluid level for the fluid monitoring device of FIG. 2A for monitoring different fluids. While the measured plots vary according to different fluids contained in the fluid container, each plot 202a, 202b and 202c can be calibrated by using the respective calibration segments 201a, 201b and 201c. During the calibration, dielectric-property related information of the respective fluids inside the fluid container can be determined and used to calibrate the respective measurement segments. After the calibration, the fluid level or volume of the various fluids can be determined from the respective measurement segments of the plots.

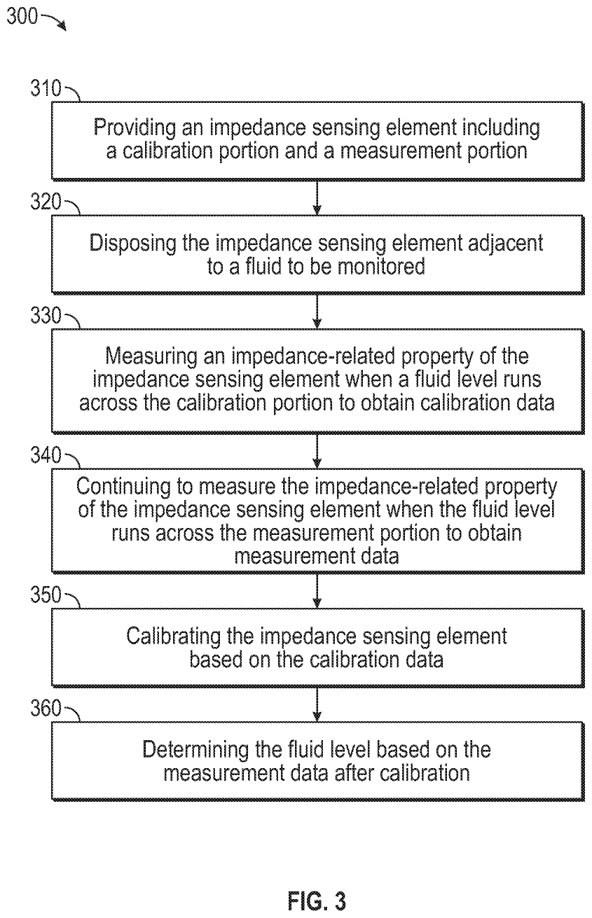

FIG. 3 illustrates a flow diagram of a self-calibration process 300 to determine a fluid level of an unknown fluid in a fluid container. At 310, an impedance sensing element including a calibration portion and a measurement portion is provided. The impedance sensing element can be for example, the impedance sensing element 210 of FIG. 2A including the calibration portion 212 and the measurement portion 214 electrically connected to each other. The process 300 then proceeds to 320.

At 320, the impedance sensing element is disposed adjacent to a fluid to be monitored. In some embodiments, the impedance sensing element can be disposed on an outside of a fluid container, for example, an infusion line or a fluid bag of an intravenous (IV) therapy. The process 300 then proceeds to 330.

At 330, an impedance-related property of the impedance sensing element is measured when a fluid level runs across the calibration portion to obtain calibration data. In the depicted embodiment of FIGS. 2A-C, when the fluid level B runs across the calibration portion 212 of the impedance sensing element 210, the impedance-related property of the impedance sensing element 210 is measured to obtain the calibration segment 201 as shown in the plot of FIG. 2B. The process 300 then proceeds to 340.

At 340, when the fluid level runs across the measurement portion, the impedance sensing element continues to measure the impedance-related property to obtain measurement data. In the depicted embodiment of FIGS. 2A-C, when the fluid level B runs across the measurement portion 214 of the impedance sensing element 210, the impedance-related property of the impedance sensing element 210 is measured to obtain the measurement segment 202 as shown in the plot of FIG. 2B. The process 300 then proceeds to 350.

At 350, the impedance sensing element is calibrated, via a circuit unit or a microprocessor, based on the calibration data. In some embodiments, the slopes of a calibration segment (e.g., S1 of segment 201 in FIG. 2B) and a measurement segment (e.g., S2 of segment 202 in FIG. 2B) can be determined, respectively, from a measured plot. While the slopes S1 and S2 each may vary upon different fluids in a fluid container, the measurement data can be calibrated by the calibration data to be independent from dielectric properties of the fluids to be monitored. For example, in some embodiments, the ratio of S1 and S2 may be a constant, which can be utilized to calibrate the measurement data. The process 300 then proceeds to 360.

At 360, the fluid level of the fluid to be monitored is determined, via the circuit unit, based on the measurement data with the calibration at 350. It is to be understood that the impedance-related property of the sensing element may linearly or non-linearly vary with the fluid level (or fluid volume). Such a linear or non-linearly relationship can be used to calibrate the measurement data and determine various fluid properties (e.g., a fluid volume, a fluid level, a fluid flow rate, etc.) based on the calibrated measurement data.

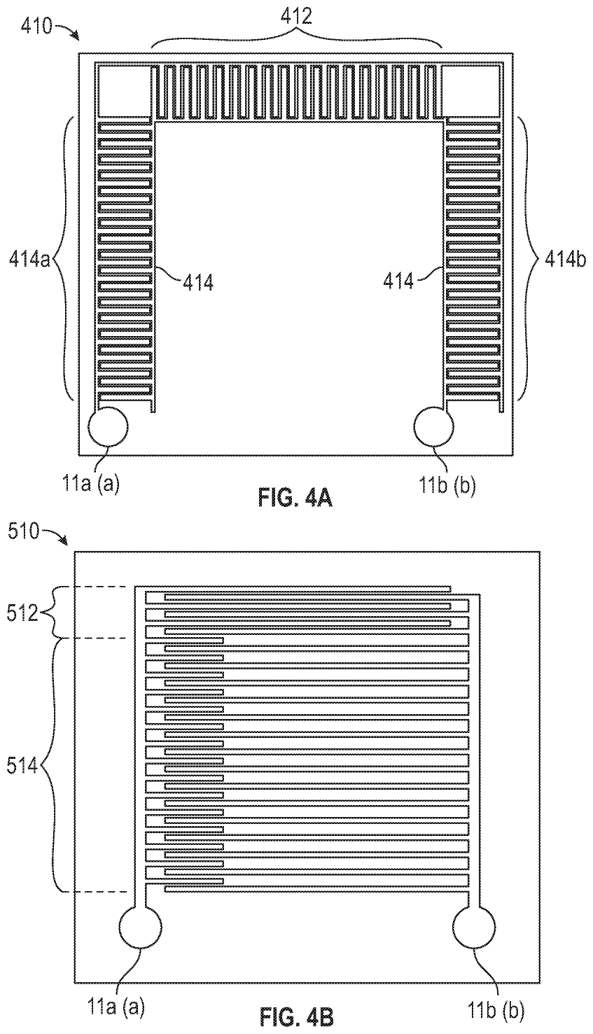

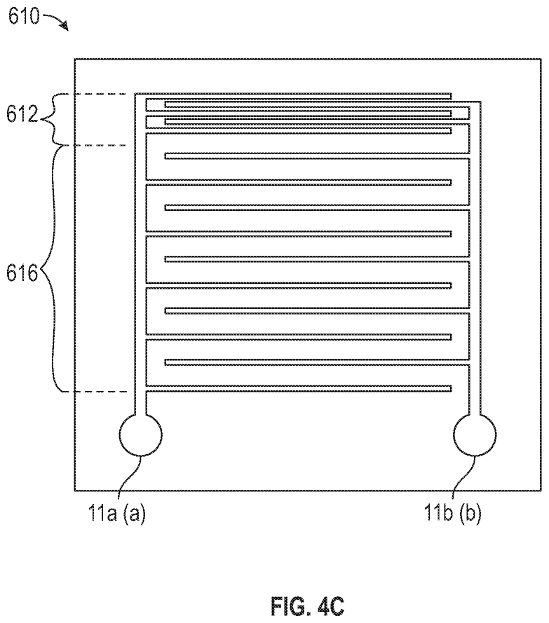

The impedance sensing elements described herein may have various configurations and can be utilized to implement a self-calibration process such as the method 300 to determine various fluid properties (e.g., a fluid volume, a fluid level, a fluid flow rate, etc.) of an unknown fluid. FIGS. 4A-C illustrate exemplary sensing elements 410, 510 and 610 each including a calibration portion and a measurement portion, according to some embodiments. The impedance sensing elements 410, 510 and 610 each include a pairing of interdigitated electrode or finger arrays a and b connected to the connection pads 11a and 11b, respectively. The fingers a and b are arranged interdigitated and parallel with respect to each other.

In the embodiment of FIG. 4A, the impedance sensing element 410 includes the calibration portion 412 and the measurement portion 414 having different orientations. The calibration portion 412 includes an array of interdigitated fingers extending along a horizontal direction. The measurement portion 414 includes a first array of interdigitated fingers 414a and a second array of interdigitated fingers 414b each extending along a vertical direction. The first and second arrays 414a-b are electrically connected to opposite ends of the calibration portion 412 to form an up-side-down “U” shape. Such a difference in the finger orientation can attribute to different slopes in the corresponding plot of impedance versus fluid level such as the plot shown in FIG. 2B.

In the embodiment of FIG. 4B, the impedance sensing element 510 includes the calibration portion 512 and the measurement portion 514 electrically connected with each other. The calibration portion 512 and the measurement portion 514 form an array of interdigitated fingers extending along a vertical direction. The calibration portion 512 and the measurement portion 514 have different configurations. That is, the interdigitated fingers a of the calibration portion 512 connected to the connection pad 11a has a finger length greater than that of the measurement portion 514. Such a difference in the finger length can attribute to different slopes in the corresponding plot of admittance, capacitance, or conductance versus fluid level such as the plot shown in FIG. 2B.

In the embodiment of FIG. 4C, the impedance sensing element 610 includes the calibration portion 612 and the measurement portion 614 electrically connected with each other. The calibration portion 612 and the measurement portion 614 form an array of interdigitated fingers extending along a vertical direction. The calibration portion 612 and the measurement portion 614 have different configurations. That is, the interdigitated fingers of the calibration portion 612 has a finger density greater than that of the measurement portion 614, i.e., the distance between the fingers is greater for the measurement portion 614 than for the calibration portion 612. Such a difference in the finger density can attribute to different slopes in the corresponding plot of admittance, capacitance, or conductance versus fluid level such as the plot shown in FIG. 2B.

While FIGS. 4A-C illustrate various impedance sensing elements having exemplary configurations, it is to be understood that any desired configuration can be used as long as the corresponding calibration portion and measurement portion have a difference such as to attribute to different slopes in the corresponding plot of admittance, capacitance, or conductance versus fluid level such as the plot shown in FIG. 2B.

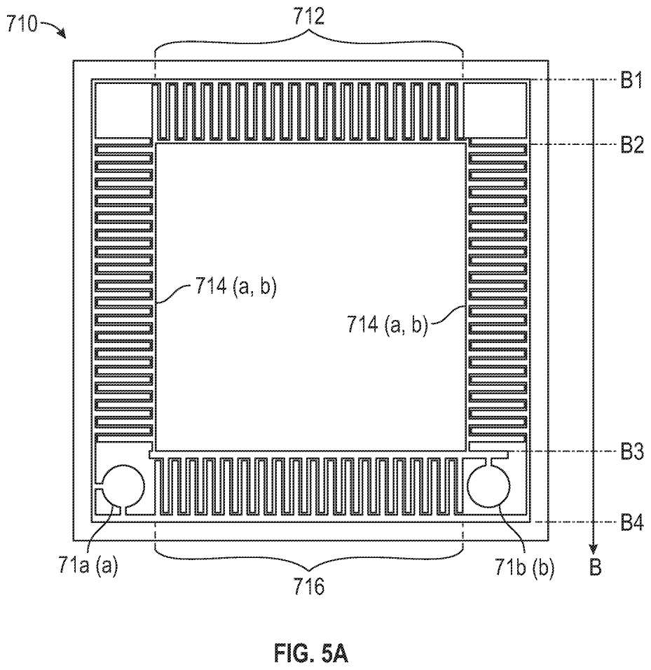

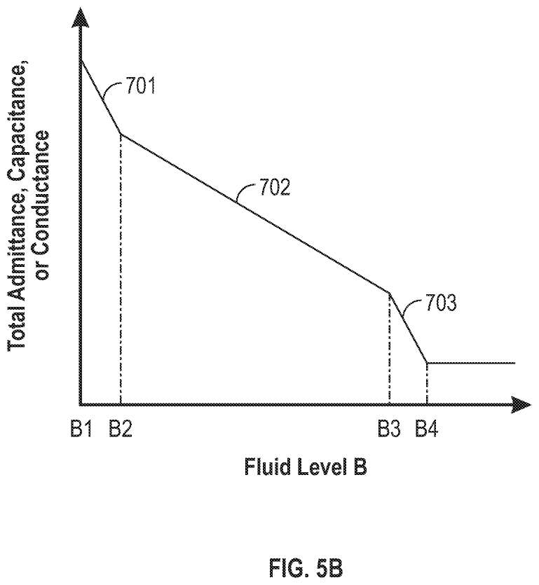

FIG. 5A illustrates a schematic side view of an impedance sensing element 710, according to one embodiment. The impedance sensing element 710 includes a calibration portion 712, a measurement portion 714 and a bottom portion 716 electrically connected to each other to form a paring of finger arrays a and b connected to connection pads 71a and 71b, respectively. The finger arrays are arranged in a rectangular shape. The calibration portion 712 includes an array of interdigitated fingers extending along a horizontal direction to form an upper side of the rectangular shape. The measurement portion 714 includes a first array of interdigitated fingers 714a and a second array of interdigitated fingers 714b each extending along a vertical direction to form left and right sides of the rectangular shape. The bottom portion 716 includes an array of interdigitated fingers extending along a horizontal direction to form a lower side of the rectangular shape.

An impedance-related property (e.g., impedance, admittance, capacitance, conductance, etc.) of the sensing element 710 can be measured upon the variation of fluid level B along the vertical direction 5. FIG. 5B illustrates a plot of admittance, capacitance, or conductance versus fluid level for the impedance sensing element 710 of FIG. 5A that is attached to an outside of a fluid container. When the fluid level B runs across the calibration portion 712, i.e., changes from the position B1 to the position B2, the capacitance of the sensing element 710 decreases accordingly. The segment 701 of the plot between positions B1 and B2 corresponds to the calibration portion 712 of the impedance sensing element 710 and has a slope S1. When the fluid level B continues to run across the measurement portion 714, i.e., changes from the position B2 to the position B3, the capacitance of the sensing element 710 continues to decrease accordingly. The segment 702 of the plot between positions B2 and B3 corresponds to the measurement portion 714 of the impedance sensing element 710 and has a slope S2. When the fluid level B continues to run across the bottom portion 716, i.e., changes from the position B3 to the position B4, the capacitance of the sensing element 710 continues to decrease accordingly. The segment 703 of the plot between the positions B3 and B4 corresponds to the bottom portion 716 of the impedance sensing element 710 and has a slope S3.

For a given fluid to be measured, the respective slopes S1, S2 and S3 of the segments 701, 702 and 703 may be determined by the configurations of the respective portions 712, 714 and 716. In the depicted embodiment, the portions 712 and 716 have the same orientations and produce segments having substantially the same slopes (e.g., S1=S3); the portions 712/716 and 714 have different orientations and produce segments having different slopes (e.g., S1 or S3 greater than S2).

The fluid level or volume in the fluid container can be determined in real time based on a plot of impedance-related property (e.g., impedance, admittance, capacitance, conductance, etc.) versus fluid level, where the plot has a calibration segment and a measurement segment such as, for example, the plot of FIG. 5B. In some embodiments, a fluid monitoring device including impedance sensing element 710 can be calibrated by using the calibration segment 701 of the plot corresponding to the calibration portion 712 of the impedance sensing element 710. During the calibration, the dielectric property of the fluid inside the fluid container can be determined. With the calibration, the fluid level or volume in the fluid container can be determined in real time by using the measurement segment 702 of the plot when the fluid level B runs across the measurement portion 714.

When the fluid level reaches the position B3, a transitional point between the measurement portion 714 and the bottom portion 716, and runs across the bottom portion 716, the fluid monitoring device can detect the change of slopes from S2 to S3 and generate desired signals such as, for example, a warning signal.

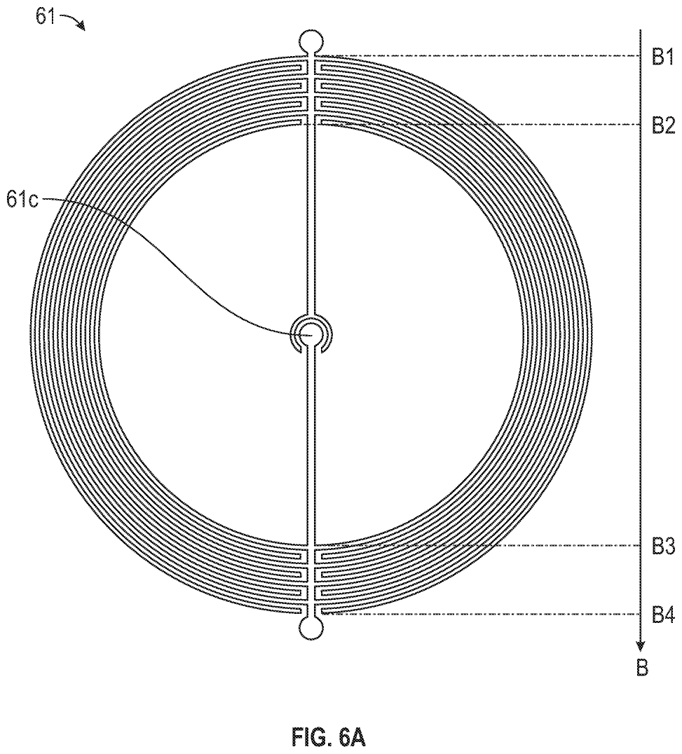

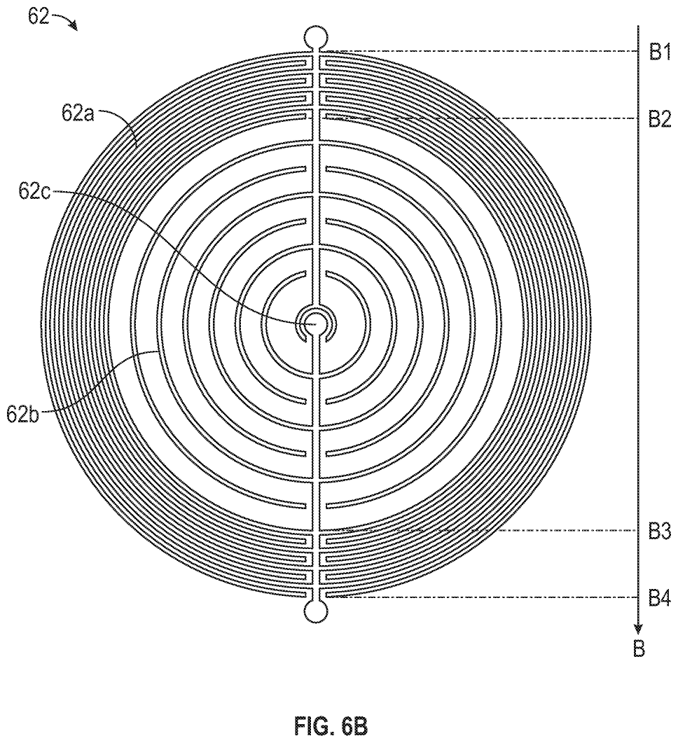

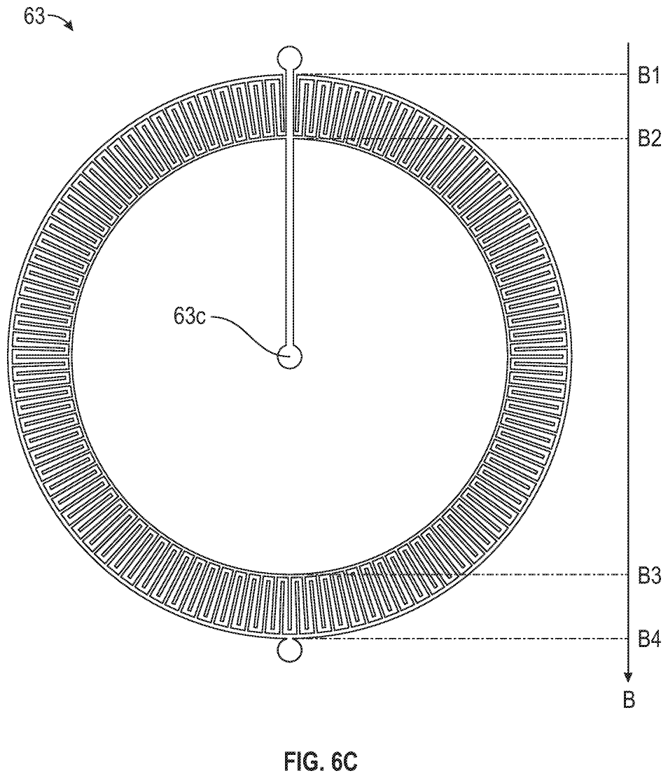

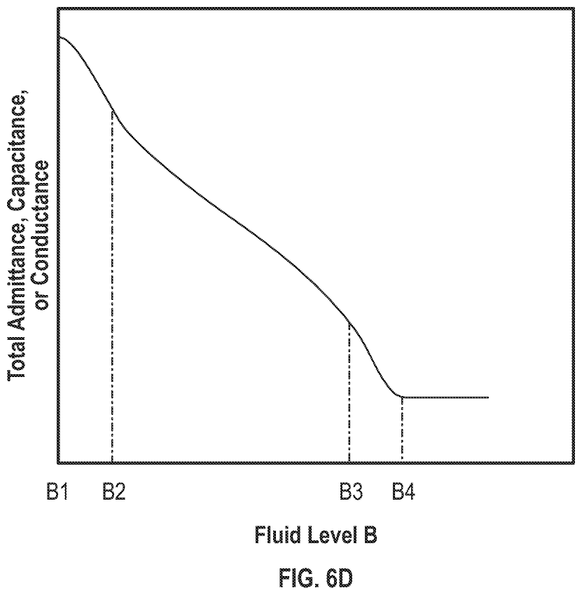

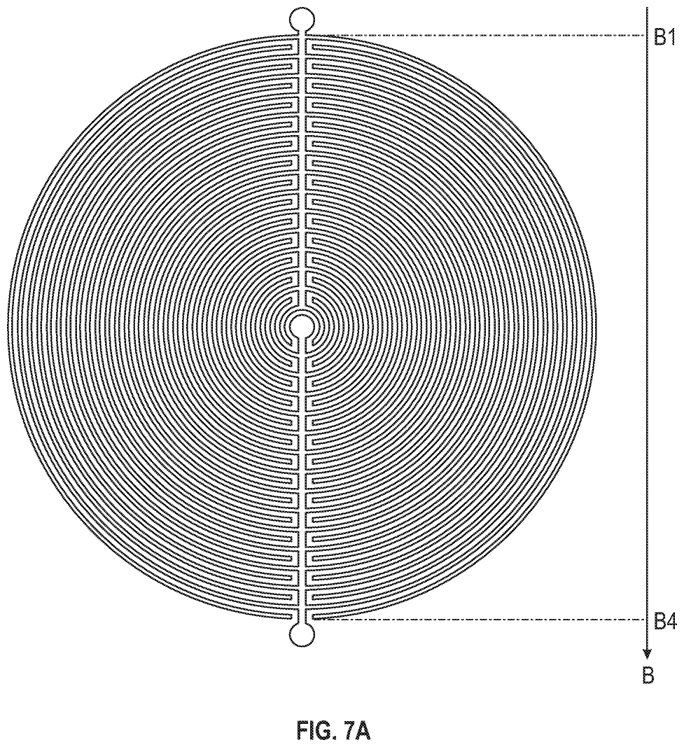

In some embodiments, an impedance sensing element described herein may have a symmetric configuration. In the embodiment depicted in FIG. 6A, the impedance sensing element 61 is a ring-shaped interdigitate capacitor which has a rotational symmetry about its center point 61. The portion of the capacitor 61 between the positions B1 and B2 corresponds to a calibration portion such as, for example, the calibration portion 712 of FIG. 5A; the portion between the positions B2 and B3 corresponds to a measurement portion such as, for example, the measurement portion 714 of FIG. 5A; the portion between the positions B3 and B4 corresponds to a bottom portion such as, for example, the bottom portion 716 of FIG. 5A. FIG. 6D illustrates a plot of admittance, capacitance, or conductance versus fluid level for the impedance sensing element 61 of FIG. 6A that is attached to an outside of a fluid container. The plot of FIG. 6D is similar to the plot of FIG. 5B and can be explained, interpreted, and processed similarly, including the transitional points B2 and B3.

In the embodiment depicted in FIG. 6B, the impedance sensing element 62 includes interdigitated electrodes or fingers arranged as an outer portion 62a and an inner portion 62b having a less finger density compared to the outer portion 62. The impedance sensing element 62 has a rotational symmetry about its center point 62c. Similar to the impedance sensing element 61, the impedance sensing element 62 has a calibration portion between points B1 and B2, a measurement portion between B2 and B3, and a bottom portion between B3 and B4, where B2 and B4 are transitional portions between two adjacent segments having different slopes. The impedance sensing element 62 can exhibit similar impedance-related properties such as showing in the plot of FIG. 6D.

In the embodiment depicted in FIG. 6C, the impedance sensing element 63 is a variant of the impedance sensing element 61. The conductors or fingers of impedance sensing element 61 are arranged radially while the fingers of the impedance sensing element 63 are arranged axially. Similar to the impedance sensing element 61, the impedance sensing element 63 has a calibration portion between points B1 and B2, a measurement portion between B2 and B3, and a bottom portion between B3 and B4, where B2 and B4 are transitional portions between two adjacent segments having different slopes. The impedance sensing element 62 can exhibit similar impedance-related properties such as showing in the plot of FIG. 6D. It is to be understood that the impedance sensing elements 61-63 have connection pads connecting to the respective interdigitated electrodes or fingers.

An impedance sensing element with a symmetric configuration can exhibit certain orientation-independency. For example, its impedance measurement can be independent from its orientation with respective to its center point (e.g., 61c, 62c, or 63c in FIGS. 6A-6C). Practically, when the impedance sensing element 61, 62, or 63 is disposed on an outside of a fluid container, no matter what the orientation is, a measured plot can be substantially the same as the plot shown in FIG. 6D, which is independent from the disposed orientation.

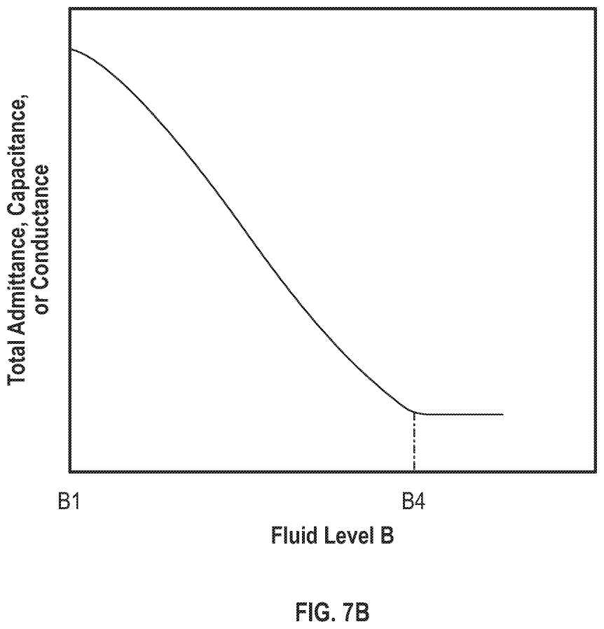

It is to be understood that an impedance sensing element can have any suitable symmetric configuration as long as the corresponding plots of impedance (admittance capacitance, conductance, etc.) versus fluid level can exhibit at least one transitional position (e.g., B2 in FIGS. 5B and 6D) between the adjacent segments having different slopes for a calibration portion and a measurement portion. Suitable symmetric configurations include, for example, ring shapes, round shapes, polygon shapes, etc., having a rotational symmetry. It is to be understood that some symmetric configurations may not have a calibration portion and a measurement portion, and the corresponding plot may not have such transitional positions therebetween, such as shown in FIGS. 7A-B.

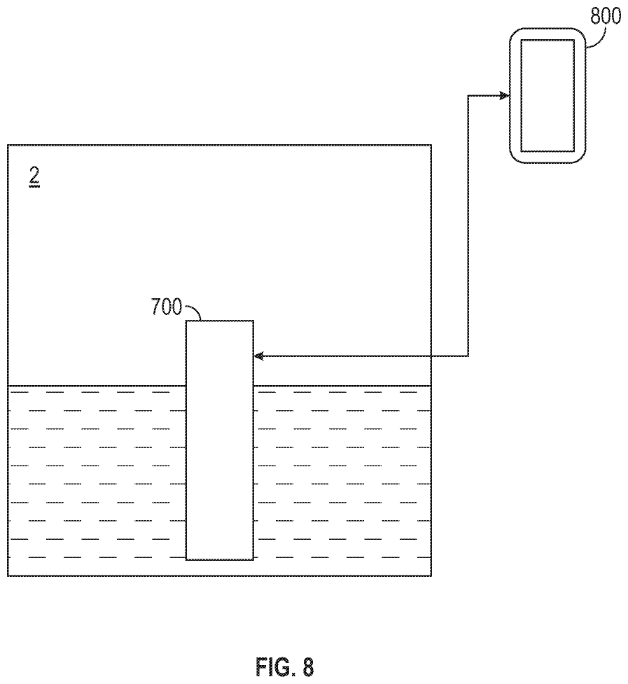

FIG. 8 illustrates a schematic diagram of a fluid monitoring device wirelessly connected to a mobile device, according to one embodiment. A fluid monitoring device 700 is attached to an outside of a fluid container 2. The fluid monitoring device 700 can include an impedance sensing element described herein. The mobile device 800 may include a wireless component that can work with the wireless component of fluid monitoring device 700 for data transmission between the mobile device 800 and the fluid monitoring device 700. The mobile device 800 can further include a graphical user interface (GUI) that is executed by a processor and displayed by a display thereof. In some embodiments, the GUI of the mobile device 800 can be provided as a mobile app that runs on the mobile device, e.g., a smart phone. The mobile app can be a computer program in any suitable programming language (e.g., Python) designed to be executed by the processor of the mobile device. The processor of the mobile device may include, for example, one or more general-purpose microprocessors, specially designed processors, application specific integrated circuits (ASIC), field programmable gate arrays (FPGA), a collection of discrete logic, and/or any type of processing device capable of executing the techniques described herein. The mobile device may also include a memory to store information. The memory can store instructions for forming the methods or processes (e.g., a self-calibration and measurement process) described herein. The memory can also store data related to the fluid monitoring device. It is to be understood that in some embodiments, the mobile device can be integrated with the fluid monitoring device 700 to be a single device in the form of, for example, a re-usable smart fluid monitoring device.

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

Listing of Exemplary Embodiments

Exemplary embodiments are listed below. It is to be understood that any one of embodiments 1-12 and 13-20 can be combined.

Embodiment 1 is a flexible sensor for fluid monitoring comprising:

a flexible substrate having a first side and a second side opposite the first side;

an impedance sensing element disposed on the first side of the flexible substrate; and

a circuit unit functionally connected to the sensing element to receive data related to an impedance of the impedance sensing element from the impedance sensing element and process the data,

wherein the impedance sensing element includes a calibration portion and a measurement portion electrically connected to the calibration portion, the calibration portion configured to generate calibration data, and the measurement portion configured to generate measurement data, and

wherein the circuit unit is configured to calibrate the measurement data based on the calibration data.

Embodiment 2 is the sensor of embodiment 1, further comprising an optional encapsulating layer and an adhesive layer disposed on the first side of the flexible substrate.
Embodiment 3 is the sensor of embodiment 2, further comprising a shielding layer disposed on the second side of the flexible substrate.
Embodiment 4 is the sensor of any one of embodiments 1-3, wherein the impedance sensing element includes an array of interdigitated electrodes.
Embodiment 5 is the sensor of embodiment 4, wherein the calibration portion includes a first portion of the interdigitated electrodes, and the measurement portion includes a second portion of the interdigitated electrodes.
Embodiment 6 is the sensor of embodiment 5, wherein the first and second portions are oriented substantially orthogonal with respect to each other.
Embodiment 7 is the sensor of embodiment 5 or 6, wherein the first and second portions have different configurations.
Embodiment 8 is the sensor of any one of embodiments 1-7, wherein the calibration portion and the measurement portion have different configurations to generate the respective adjacent segments of impedance-related property versus fluid level, the segments having different slopes.
Embodiment 9 is the sensor of embodiment 8, wherein the slope of a calibration segment is greater than that of a measurement segment.
Embodiment 10 is the sensor of any one of embodiments 1-9, wherein the impedance sensing element further includes a third portion configured to generate warning data, the third portion having a configuration different from the measurement portion.
Embodiment 11 is the sensor of any one of embodiments 1-10, wherein the impedance sensing element has a rotational symmetric configuration such that the generated data are substantially independent from an orientation of the impedance sensing element.
Embodiment 12 is an intravenous (IV) injection package comprising:

a fluid container to contain fluid; and

the flexible sensor of any one of embodiments 1-11 attached to an outer side of the fluid container.

Embodiment 13 is a method of monitoring fluid, the method comprising:

providing an impedance sensing element including a calibration portion and a measurement portion electrically connected to the calibration portion;

disposing the impedance sensing element adjacent to a volume of fluid to be monitored;

varying the fluid volume such that a fluid level thereof continuously runs across the calibration portion and the measurement portion of the sensor in sequence; and

measuring an impedance-related property of the impedance sensing element when varying the fluid volume to obtain a plot of impedance-related property versus fluid level,
wherein the plot has a calibration segment corresponding to the calibration portion of the sensor and a measurement segment corresponding to the measurement portion of the impedance sensing element, the calibration segment and the measurement segment are connected at a transitional point.
   Embodiment 14 is the method of embodiment 13, further comprising calibrating, via a circuit unit, the impedance sensing element based on the calibration segment of the plot.
   Embodiment 15 is the method of embodiment 14, further comprising determining, via the circuit unit, the fluid level based on the measurement segment of the plot after the calibration.
   Embodiment 16 is the method of any one of embodiments 13-15, further comprising integrating the impedance sensing element to a flexible sensor including an adhesive layer on a first side of the flexible sensor to cover the impedance sensing element.
   Embodiment 17 is the method of embodiment 16, further comprising providing a shielding layer disposed on a second side of the flexible sensor opposite the first side.
   Embodiment 18 is the method of any one of embodiments 13-17, wherein the calibration segment and the measurement segment have different slopes adjacent the transitional point.
   Embodiment 19 is the method of any one of embodiments 13-18, wherein the slope of the calibration segment is greater than that of the measurement segment.
   Embodiment 20 is the method of any one of embodiments 13-19, wherein the impedance sensing element has a rotational symmetric configuration such that the measured impedance-related property is substantially independent from an orientation of impedance sensing element with respect to the fluid level.

Reference throughout this specification to “one embodiment,” “certain embodiments,” “one or more embodiments,” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. In particular, as used herein, the recitation of numerical ranges by endpoints is intended to include all numbers subsumed within that range (e.g., 1 to 5 includes 1, 1.5, 2, 2.75, 3, 3.80, 4, and 5). In addition, all numbers used herein are assumed to be modified by the term “about.”

Furthermore, various exemplary embodiments have been described. These and other embodiments are within the scope of the following claims.

1. A flexible sensor for fluid monitoring comprising:
a flexible substrate having a first side and a second side opposite the first side;
an impedance sensing element disposed on the first side of the flexible substrate; and
a circuit unit functionally connected to the sensing element to receive data related to an impedance of the impedance sensing element from the impedance sensing element and process the data,
wherein the impedance sensing element includes a calibration portion and a measurement portion electrically connected to the calibration portion, the calibration portion configured to generate calibration data, and the measurement portion configured to generate measurement data, and
wherein the circuit unit is configured to calibrate the measurement data based on the calibration data.

2. The sensor of claim 1, further comprising an adhesive layer disposed on the first side of the flexible substrate.

3. The sensor of claim 2, further comprising a shielding layer disposed on the second side of the flexible substrate.

4. The sensor of claim 1, wherein the impedance sensing element includes an array of interdigitated electrodes.

5. The sensor of claim 4, wherein the calibration portion includes a first portion of the interdigitated electrodes, and the measurement portion includes a second portion of the interdigitated electrodes.

6. The sensor of claim 5, wherein the first and second portions are oriented substantially orthogonal with respect to each other.

7. The sensor of claim 5, wherein the first and second portions have different configurations.

8. The sensor of claim 1, wherein the calibration portion and the measurement portion have different configurations to generate the respective adjacent first and second segments of impedance-related property versus fluid level, the first and second segments having different slopes.

9. The sensor of claim 8, wherein the slope of the first segment of the calibration portion is greater than that of the second segment of the measurement portion.

10. The sensor of claim 1, wherein the impedance sensing element further includes a third portion configured to generate warning data, the third portion having a configuration different from the measurement portion.

11. The sensor of claim 1, wherein the impedance sensing element has a rotational symmetric configuration such that the generated data are substantially independent from an orientation of the impedance sensing element.

12. An intravenous (IV) injection package comprising:
a fluid container to contain fluid; and
the flexible sensor of claim 1 attached to an outer side of the fluid container.

13. A method of monitoring fluid, the method comprising:
providing an impedance sensing element including a calibration portion and a measurement portion electrically connected to the calibration portion;
disposing the impedance sensing element adjacent to a volume of fluid to be monitored;
varying the fluid volume such that a fluid level thereof continuously runs across the calibration portion and the measurement portion of the sensor in sequence; and
measuring an impedance-related property of the impedance sensing element when varying the fluid volume to obtain a plot of impedance-related property versus fluid level,
wherein the plot has a calibration segment corresponding to the calibration portion of the sensor and a measurement segment corresponding to the measurement portion of the impedance sensing element, the calibration segment and the measurement segment are connected at a transitional point.

14. The method of claim 13, further comprising calibrating, via a circuit unit, the impedance sensing element based on the calibration segment of the plot.

15. The method of claim 14, further comprising determining, via the circuit unit, the fluid level based on the measurement segment of the plot after the calibration.

16. The method of claim 13, further comprising integrating the impedance sensing element to a flexible sensor including an adhesive layer on a first side of the flexible sensor to cover the impedance sensing element.

17. The method of claim 16, further comprising providing a shielding layer disposed on a second side of the flexible sensor opposite the first side.

18. The method of claim 13, wherein the calibration segment and the measurement segment have different slopes adjacent the transitional point.

19. The method of claim 13, wherein the slope of the calibration segment is greater than that of the measurement segment.

20. The method of claim 13, wherein the impedance sensing element has a rotational symmetric configuration such that the measured impedance-related property is substantially independent from an orientation of impedance sensing element with respect to the fluid level.
